(19) United States
(12) Patent Application Publication
(10) Pub. No.: US 2014/0107518 A1
(43) Pub. Date: Apr. 17, 2014

(54) RESUSCITATION DEVICE

(71) Applicant: CAREFUSION 207, INC., San Diego, CA (US)

(72) Inventor: Neil KORNEFF, Diamond Bar, CA (US)

(73) Assignee: CAREFUSION 207, INC., San Diego, CA (US)

(21) Appl. No.: 13/650,037

(22) Filed: Oct. 11, 2012

(51) Int. Cl. A61M 16/20; A61B 5/087; A61M 16/08

(52) U.S. Cl. 600/538; 128/205.13

(57) ABSTRACT

A resuscitation device comprising a main housing, and an expiratory relief valve.

### BACKGROUND

Resuscitation devices are utilized to facilitate in resuscitating a patient.

### BRIEF DESCRIPTION OF THE DRAWINGS

FIGS. 1A-4 and 6A-9 illustrate embodiments of various resuscitation devices.

FIG. 5 depicts an embodiment of a manometer sub-assembly.

The drawings referred to in this description should be understood as not being drawn to scale except if specifically noted.

### BRIEF DESCRIPTION

Reference will now be made in detail to embodiments of the present technology, examples of which are illustrated in the accompanying drawings. While the technology will be described in conjunction with various embodiment(s), it will be understood that they are not intended to limit the present technology to these embodiments. On the contrary, the present technology is intended to cover alternatives, modifications and equivalents, which may be included within the spirit and scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

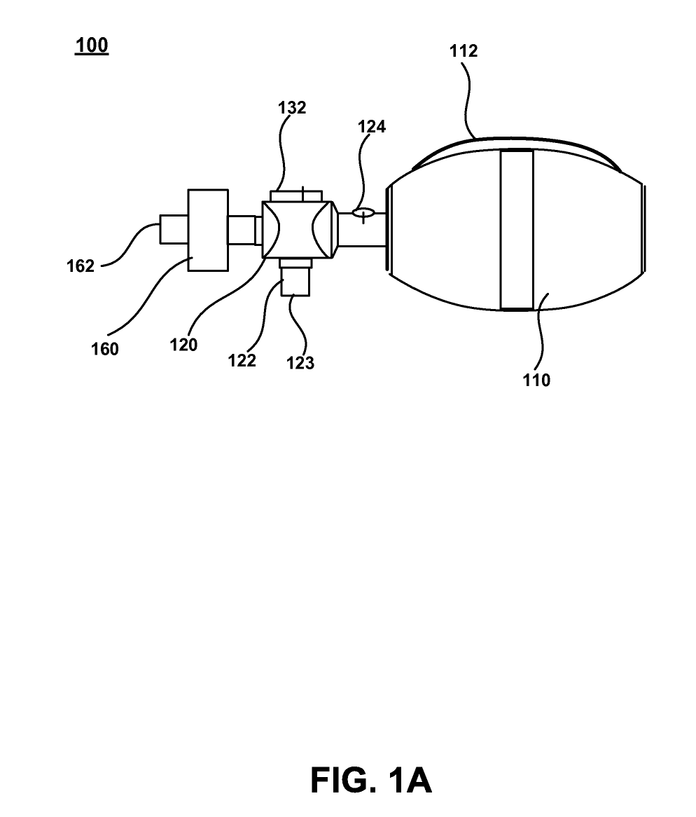

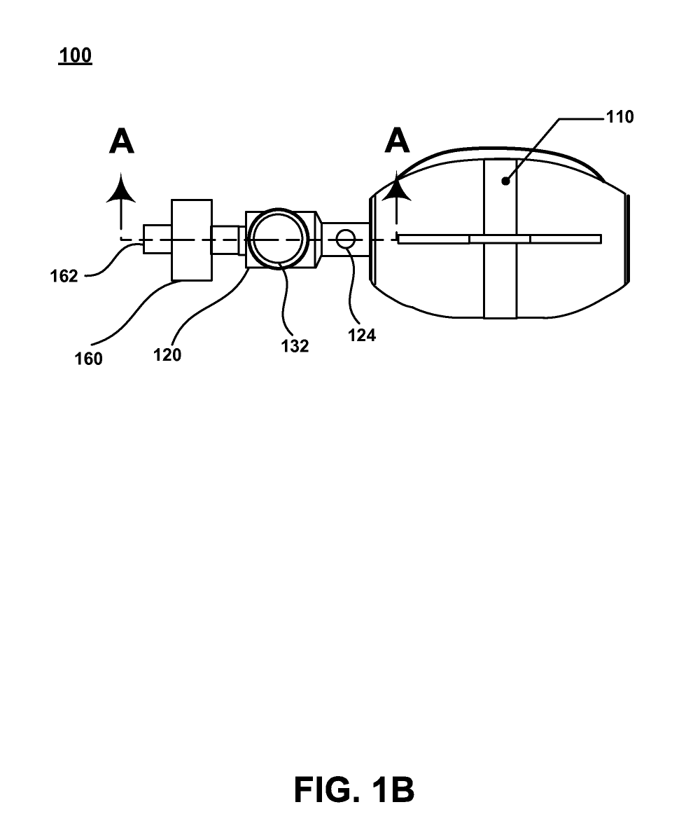

FIGS. 1A-B depicts a side view and top view, respectively, of resuscitation device 100. In general, resuscitation device 100 for facilitating in the resuscitation of a patient, which will be described in further detail below.

Resuscitation device 100 includes, among other things, bulb 110, pop-off valve 124, cap 132, main housing 120 and exhalation filter 160.

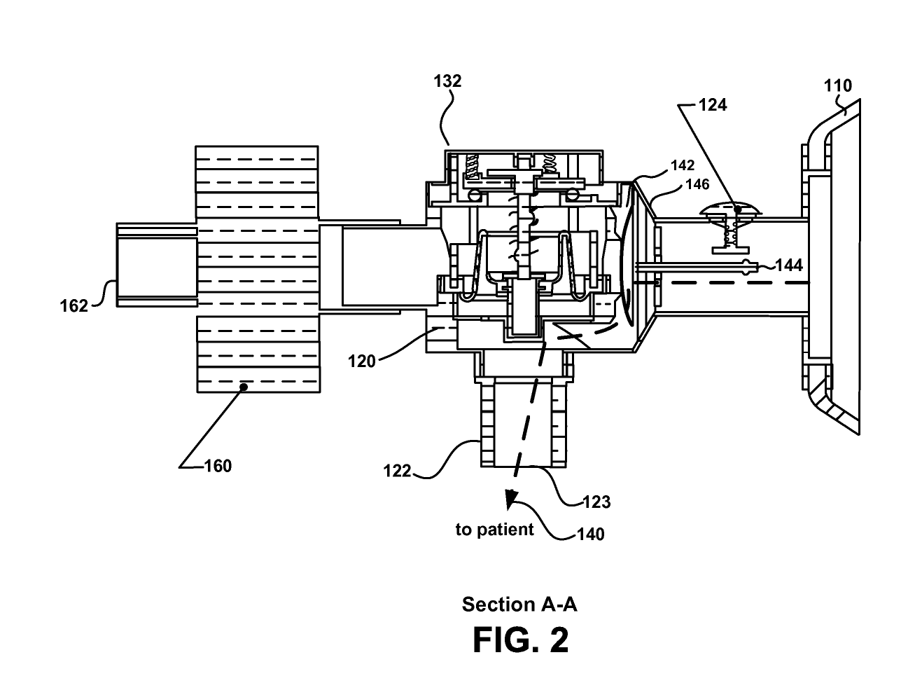

FIG. 2 depicts the cross-sectional view of resuscitation device 100 along section A-A during the inhalation period. In order to facilitate a forced inhalation for a patient, bulb 110 is manually compressed or squeezed. The compression of the bulb generates an inhalation airflow which travels along inhalation airflow path 140. In particular, the inhalation airflow provides sufficient pressure to unseat valve flap 142 (which is attached to central pin 144) from valve seat 146 such that the inhalation airflow travels between valve flap 142 and valve seat 146. Accordingly, inhalation airflow travels along inhalation airflow path 140 through housing 120 (and pipe stub 122) and finally to a patient via a mask, tube or other interface.

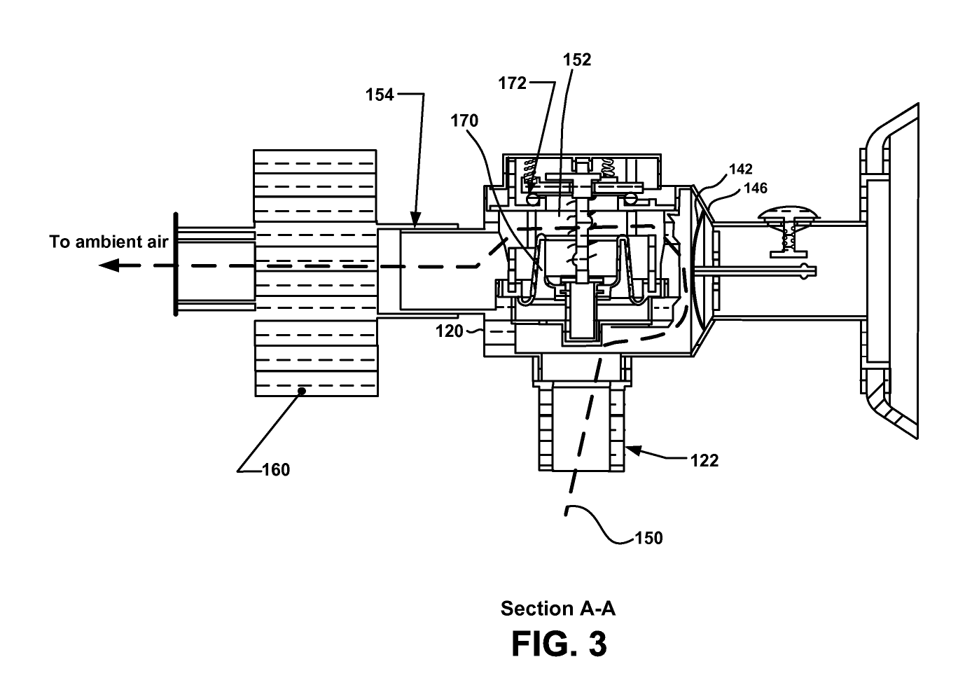

FIG. 3 depict a cross-sectional view of resuscitation device 100 along section A-A during the exhalation period. After the inhalation airflow is generated by the squeezing of bulb 110, a caregiver releases (or stops compressing) bulb 110. Bulb 110 then resiliently re-inflates to its original form. The re-inflation causes a vacuum inside of bulb 110. Consequently, the vacuum pulls or translates the valve flap 142 and the central pin 144 towards bulb 110 such that valve flap 142 reseals with valve seat 146. Accordingly, the exhaled breath of the patient is directed along exhalation airflow path 150 through pipe stub 122, exhalation pathway 152, exhalation port 154 and exhalation filter 160.

It is noted that exhalation pathway 152 is a portion of housing 120 that allows for the passage of exhalation airflow and includes at least the area located above diaphragm 170 and below sealing ring 172.

It is often desirable to filter the expiratory gasses from a patient. For example, it is desirable to filter expiratory gasses when the patient is known to have an illness that is easily transmitted. Placing a filter on a resuscitation bag is not common in conventional resuscitation bags because the resuscitation bag will become inoperable if the filter is clogged.

As described above, during exhalation, the exhalation gasses are directed along exhalation airflow path 150 through exhalation port 154 and exhalation filter 160. Exhalation filter 160 is utilized to filter the exhalation gasses prior to the exhalation airflow exiting into ambient air. Exhalation filter 160 can be integral or removable with resuscitation device 100.

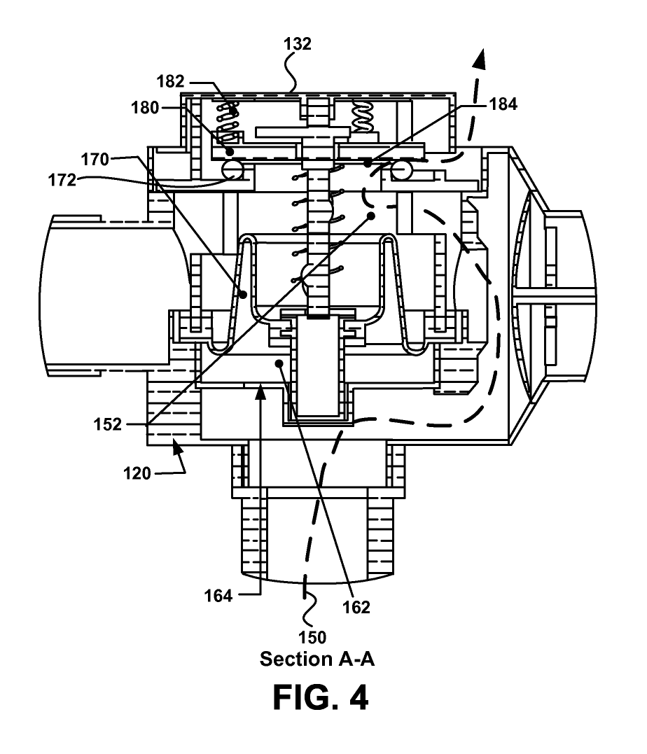

FIG. 4 depicts an embodiment of exhalation airflow path 150 in response to exhalation filter 160 being clogged and/or providing too high of a resistance. In the event that exhalation filter 160 is clogged or provides too high of a resistance, the pressure in housing 120 (e.g., pressure in exhalation pathway 152) increases and is higher than normal. As a result, the increased pressure pushes up against exhalation or expiratory relief valve 180 (and compresses exhalation relief springs 182) and unseats exhalation relief valve 180 from sealing ring 172. Accordingly, a gap is created between exhalation relief valve 180 from sealing ring 172 such that exhalation airflow path 150 travels between exhalation relief valve 180 from sealing ring 172 to ambient air.

It is desirable to determine if there is carbon dioxide in the exhaled gasses. This provides a level of confidence to the caregiver that the patient is being properly ventilated.

Referring to FIG. 4, a colorimetric indicator 184 is affixed to the bottom of exhalation relieve valve 180. The sealing ring enables a protective backing paper (not shown) to cover colorimetric indicator 184 until the caregiver pulls the backing paper from the assembly.

Exhalation relief valve 180 and cap 132 are transparent enabling the colorimetric indicator 184 to be visible to the caregiver. Expiratory gasses within exhalation pathway 152 are compelled towards the colorimetric indicator 184 by the design of the flow path. In one embodiment, colorimetric indicator 184 is placed closer towards the center of exhalation pathway 152.

Referring to FIG. 2, pop-off valve 124 allows for the release of pressure within resuscitation device 100 when the pressure exceeds a designated limit. For example, pop-off valve 124 becomes unseated and relieves pressure when the pressure exceeds a designated limit.

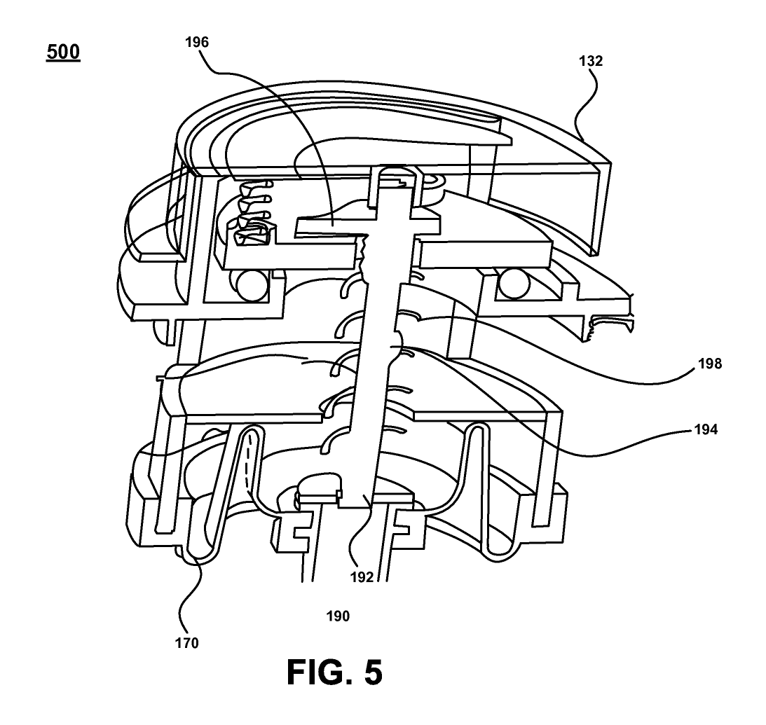

The manometer allows for airway gas pressures (exhalation and inhalation gas pressures) to be measured. The manometer may be integrated with resuscitation bag housing or may be a removable sub-assembly. FIG. 5 depicts one embodiment of manometer 500 as a removable sub-assembly.

Referring to FIGS. 4 and 5, diaphragm 170 is in a relaxed state (assuming there is no exhalation or inhalation pressure in the system). During exhalation or inhalation, a pressure (exhalation or inhalation pressure) is experienced at the bottom (i.e., the surface facing away from cap 132) of diaphragm 170. The area above diaphragm 170 (e.g., exhalation pathway 152) is at atmospheric pressure. Accordingly, when the exhalation/inhalation pressure is greater than the atmospheric pressure, the pressure difference between the area above and below diaphragm forces diaphragm 170 to move in the upward direction (i.e., towards cap 132).

The movement of diaphragm 170 upwards also moves bearing 190 up along shaft 192. A groove in bearing 190 interacts with protrusion 194 which spirally wraps around shaft 192, thereby rotating shaft 192. Thus, pointer 196, which is attached to shaft 192, is rotated to point to the associated pressure indication. Once the inhalation/exhalation pressure is dissipated, compression spring 198 urges diaphragm 170 back to its relaxed state.

FIG. 4 depicts lower chamber 162 (or pressure muting chamber) disposed below diaphragm 170. Lower chamber 162 is in fluid communication with exhalation or inhalation airflow of a patient, via lower chamber orifice 164. That is, a portion of the exhalation or inhalation airflow enters into lower chamber 162, via lower chamber orifice 164. As a result, pressure signals from the exhalation or inhalation airflow are transmitted to diaphragm 170.

Lower chamber orifice 164 is sized such that rapid changes in pressure are muted in lower chamber 162. This prevents excessive acceleration of diaphragm 170. Any volume of gas in lower chamber 162 must also exit through lower chamber orifice 164. Therefore, lower chamber 162 regulates movement of diaphragm 170 for both inhalation and exhalation.

In one embodiment, for example, an embodiment including lower chamber 162, diaphragm 170 is directly exposed to the exhalation airflow along exhalation airflow path 150.

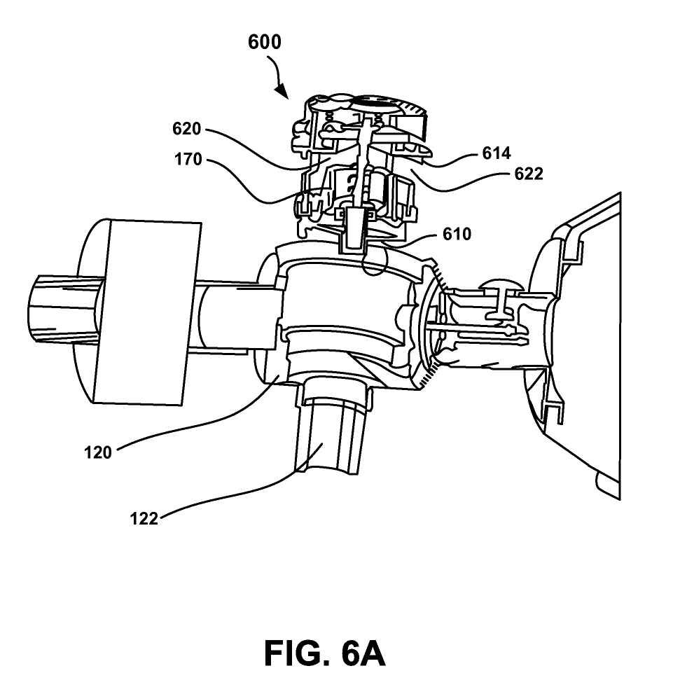

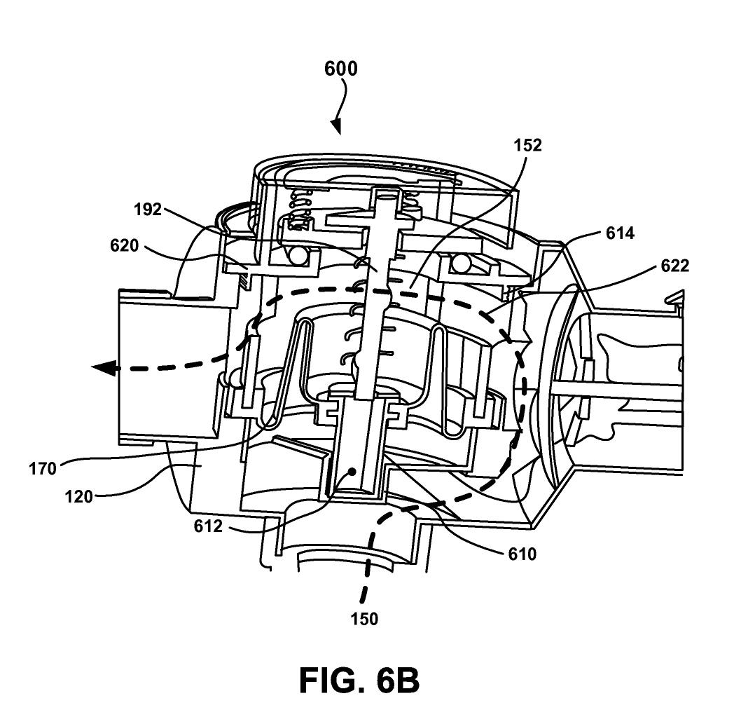

FIGS. 6A-B depicts an embodiment of manometer sub-assembly 600. Manometer sub-assembly 600 optionally includes aligning geometry 610. Aligning geometry 610 is for aligning moving portion 612. Moving portion 612 is for receiving shaft 192, when diaphragm 170 is moved upwards in response to exhalation/inhalation pressure, as described above. It is also noted that the manometer sub-assembly 600 is releasably attached to housing 120 via threads 614.

In various embodiments of manometer sub-assembly 600, the area below diaphragm 170 is completely open (e.g., does not include lower chamber 162) and the bottom portion of diaphragm 170 is directly exposed to both inhalation/exhalation gasses.

Also, the area above diaphragm 170 is substantially open. For example, upper housing 620, of manometer sub-assembly 600, includes opening 622. Opening 622 allows for exhalation airflow path 150 (as described with respect to at least FIG. 4) to travel through upper housing 620. Opening 622 must be of a substantial size to let the exhalation gasses pass through. Accordingly, upper housing 620 is a substantially open housing. It should be appreciated that opening 622 is a part of exhalation pathway 152. Moreover, the top portion of diaphragm 170 is directly exposed to the exhalation gas that travels along exhalation airflow path 150.

During the inhalation period, inhalation airflow path 140 is the same path as described with respect to FIG. 2.

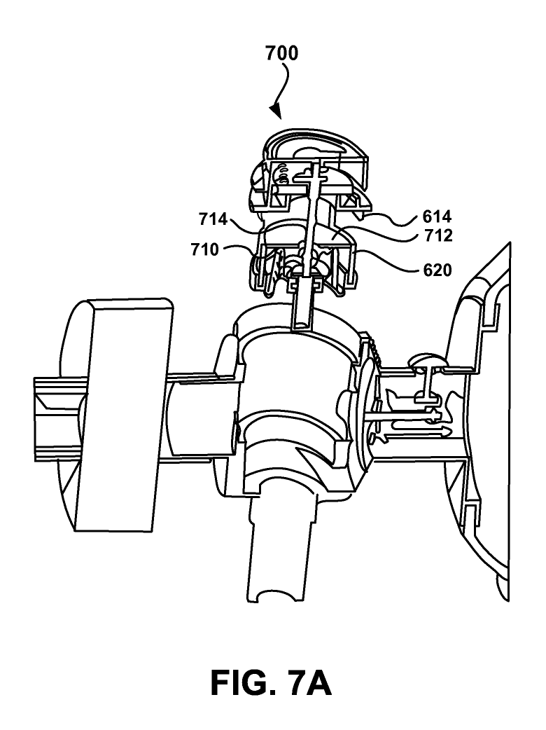

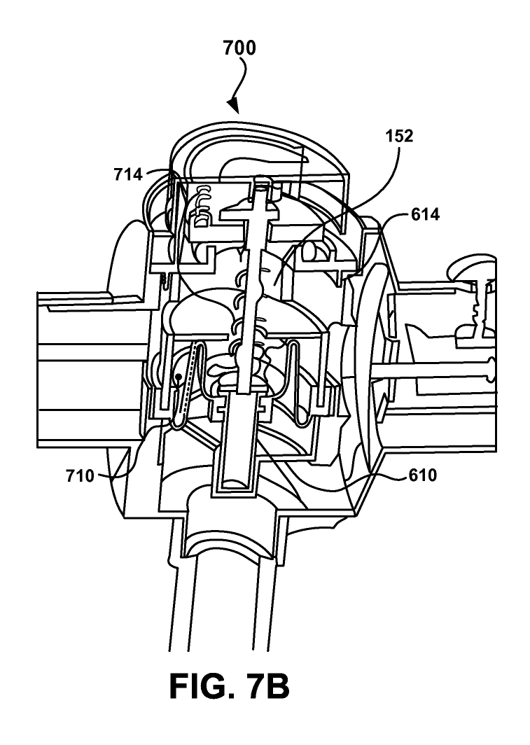

FIGS. 7A-B depicts an embodiment of manometer sub-assembly 700. Manometer sub-assembly 700 includes moving portion 612 and optionally includes aligning geometry 610. Manometer sub-assembly 700 is releasably attached to resuscitation bag housing 120.

Manometer sub-assembly 700 does not include a chamber (e.g., lower chamber 162) below diaphragm 170. For example, the area below diaphragm 170 is completely open. In particular, bottom portion of diaphragm 170 is directly exposed to the exhalation/inhalation gasses.

Manometer sub-assembly 700 does include upper chamber 710 located directly above diaphragm 170. Upper chamber 710 has a lower boundary of diaphragm 170, an upper boundary of plate 712 and a side boundary of upper housing 620.

The pressure of the exhalation gasses travelling through exhalation pathway 152 (along exhalation airflow path 150) is communicated through opening 714 to the upper portion of diaphragm 170. As such, the differential pressure between the upper and lower portion of diaphragm 170 compels its movement, as described above.

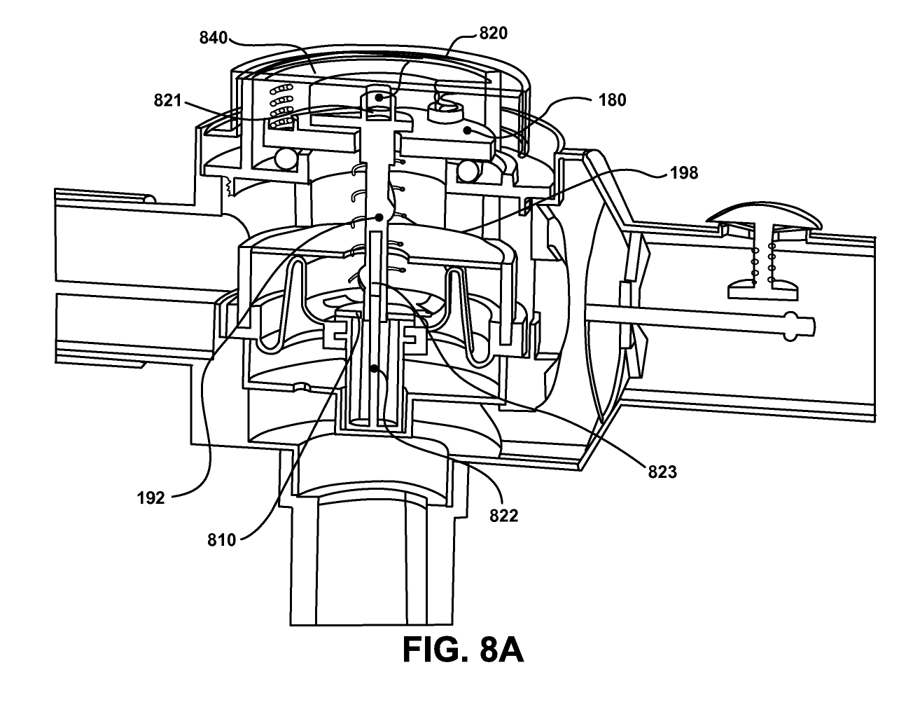

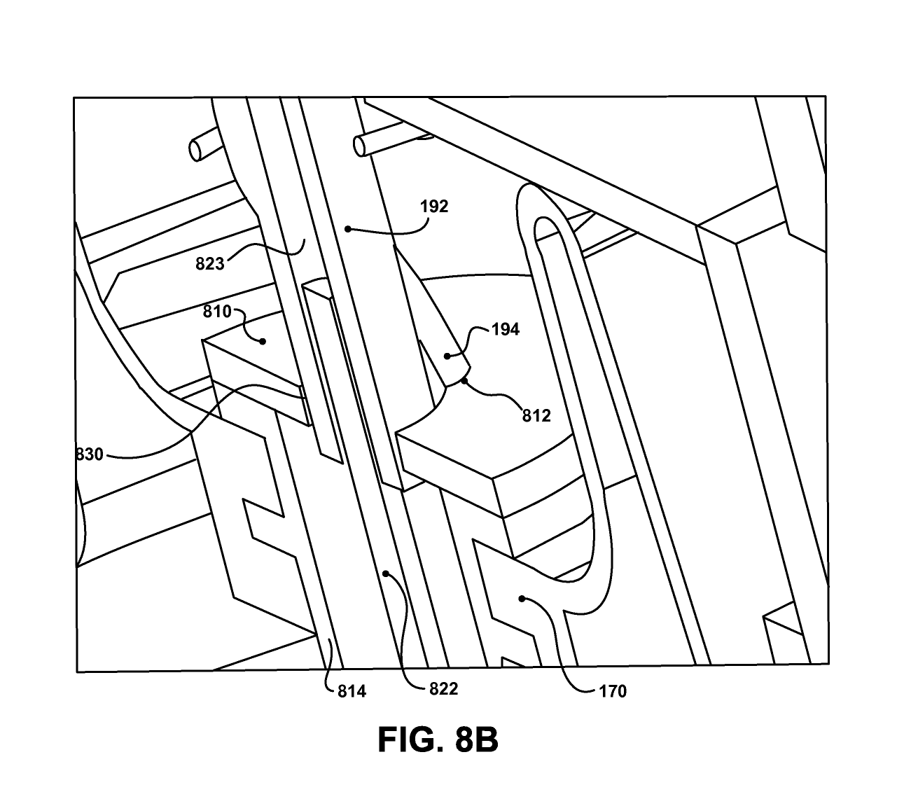

Referring now to at least FIGS. 8A-B, groove cap 810 includes groove 812 which interacts with protrusion 194, as described above. It is noted that groove cap 810 is not in direct contact with diaphragm 170. In particular, groove cap 810 is bonded to groove cap housing 814 and does not make direct physical contact with diaphragm 170.

Bearing 820 and bearing 822 index on geometry interior to helical shaft 192. For instance, bearing 822 seats in cavity 823 and bearing 820 seats in cavity 821. It is noted that shaft 192 rotates when protrusion 194 physically interacts with and translates through groove 812.

Bearing 822 provides adequate support such that helical shaft 192 does not contact groove cap 810 at any location resulting in a gap 830 between helical shaft 192 and groove cap 810.

Also, helical shaft 192 does not make contact with any other geometry other than the pivot or bearing 820 and bearing 822. For example, helical shaft 192 does not contact the exhalation relief valve 180. It is noted that various embodiments of resuscitation devices or manometer sub-assemblies, as described herein, can include the bearing configuration of bearing 820 and bearing 822.

In various embodiments, helical shaft 192 can be fabricated to have a larger diameter and a pointer is not provided (e.g., pointer 196). The media marked with indications 840 can be affixed to the larger diameter helical shaft 192 such that the indications (attached to helical shaft 192) rotate with varying pressure. Moreover, a reference point can be located on the device, such as on the exhalation relief valve 180, to provide a point to read/view the pressure values.

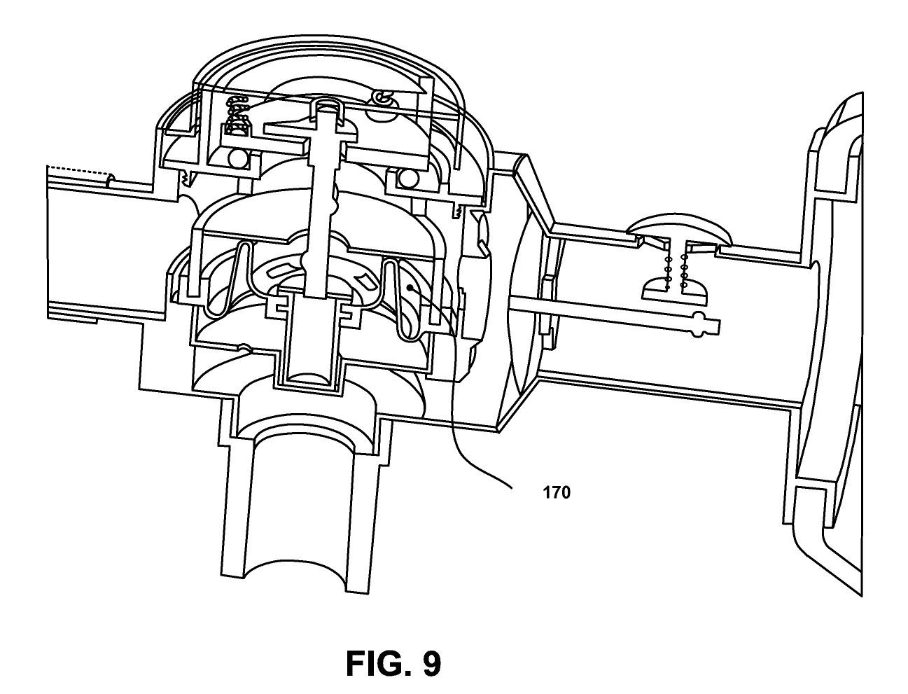

FIG. 9 depicts an embodiment of diaphragm 170 that is able to resiliently restore itself back to a rest position. Accordingly, a compression spring is not necessary or needed in the assembly. It should be appreciated that the no compression spring concept can be applied to any embodiments described herein.

Various embodiments of the present invention are thus described. While the present invention has been described in particular embodiments, it should be appreciated that the present invention should not be construed as limited by such embodiments, but rather construed according to the following claims. Moreover, various embodiments described herein may be utilized alone or in combination with one another.

1. A resuscitation device comprising:
a main housing; and
an expiratory relief valve.

2. The resuscitation device of claim 1, further comprising an expiration filter, wherein said expiration filter is disposed external said main housing.

3. The resuscitation device of claim 1, further comprising:
a carbon dioxide (CO2) detector.

4. The resuscitation device of claim 1, further comprising:
a pop-off valve configured to unseat when a pressure from a squeeze bulb exceeds a predetermined threshold.

5. The resuscitation device of claim 1, further comprising a manometer sub-assembly, wherein said manometer sub-assembly is attached to said main housing, said manometer sub-assembly comprising:
a diaphragm, wherein said diaphragm is in direct fluid communication with an inhalation pathway and an exhalation pathway.

6. The resuscitation device of claim 5, wherein said resuscitation device does not require a compression spring to urge said diaphragm to a resting position.

7. The resuscitation device of claim 5, wherein said manometer sub-assembly is removably attached to said main housing.

8. The resuscitation device of claim 5, wherein said manometer sub-assembly further comprises:
a pressure muting chamber configured to mute changes in pressure.

9. The resuscitation device of claim 5, wherein said manometer sub-assembly further comprises:
a groove cap that is not required to be in physical contact with said diaphragm.

10. The resuscitation device of claim 5, wherein said manometer sub-assembly further comprises:
a helical shaft, wherein a first bearing is journaled in a first end of said helical shaft and a second bearing is journaled in a second end of said helical shaft.

11. A resuscitation device comprising:
a main housing;
an expiration filter, wherein said expiration filter is disposed external said main housing;
a manometer releasably attached to said main housing; and
a diaphragm, wherein said diaphragm is in direct fluid communication with an inhalation pathway and an exhalation pathway.

12. The resuscitation device of claim 11, further comprising:
a carbon dioxide (CO2) detector.

13. The resuscitation device of claim 11, further comprising:
a pop-off valve configured to unseat when a pressure from a squeeze bulb exceeds a predetermined threshold.

14. The resuscitation device of claim 11, wherein said resuscitation device does not require a compression spring to urge said diaphragm to a resting position.

15. The resuscitation device of claim 11, further comprising:
an exhalation relief valve configured to unseat when an exhalation pressure exceeds a predetermined threshold.

16. The resuscitation device of claim 11, further comprising:
an exhalation relief valve spring.

17. The resuscitation device of claim 11, further comprising:
a pressure muting chamber configured to mute rapid changes in pressure.

18. The resuscitation device of claim 11, further comprising:
a groove cap that is not required to be in physical contact with said diaphragm.

19. The resuscitation device of claim 11, further comprising
a shaft, wherein a first bearing is journaled in a first end of said shaft and a second bearing is journaled in a second end of said shaft.

20. The resuscitation device of claim 11, wherein said manometer measures inhalation pressure and exhalation pressure.

21. The resuscitation device of claim 11, wherein said expiration filter is releasably attached to said main housing.
